(19) United States
(12) Patent Application Publication
(10) Pub. No.: US 2024/0358950 A1
(43) Pub. Date: Oct. 31, 2024

(54) SUPRAGLOTTIC AIRWAY (SGA) WITH TELESCOPING END FOR CAMERA

(71) Applicant: Covidien LP, Mansfield, MA (US)

(72) Inventors: Peter Douglas Colin Inglis, Boulder, CO (US); Derek S. Tata, Boulder, CO (US)

(21) Appl. No.: 18/631,986

(22) Filed: Apr. 10, 2024

Related U.S. Application Data
(60) Provisional application No. 63/498,697, filed on Apr. 27, 2023.

(51) Int. Cl. A61M 16/04; A61B 1/267

(52) U.S. Cl. CPC A61M 16/0434; A61B 1/267; A61M 16/0409; A61M 2205/583; A61M 2209/00; A61M 2210/0656

(57) ABSTRACT

The disclosed technology relates to a telescoping supraglottic airway device, such as a laryngeal mask airway. An example supraglottic airway device includes a cuff defining an orifice, a telescoping airway channel, extending in a proximal direction from the cuff, defined by first and second concentric cylinders; and a spring that biases the first and second cylinders apart to extend the telescoping airway channel.

### CROSS-REFERENCE TO RELATED APPLICATION

The present application claims the benefit of U.S. Provisional Patent Application Ser. No. 63/498,697, filed on Apr. 27, 2023, the entire content of which is incorporated herein by reference.

### INTRODUCTION

Airway management is the assessment, planning, and series of medical procedures required to maintain or restore an individual's ventilation or breathing. Airway devices play an important role in airway management by serving as the interfaces between patients and medical ventilator systems to provide ventilatory and supplemental oxygen support. Supraglottic airway devices are a type of airway device that continue to evolve and are increasingly relied upon in both emergent and non-emergent situations due to their modular structure, versatility, and less invasive nature.

It is with respect to this general technical environment that aspects of the present technology disclosed herein have been contemplated. Furthermore, although a general environment is discussed, it should be understood that the examples described herein should not be limited to the general environment identified herein.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In an aspect, the technology relates to a telescoping supraglottic airway device that includes a cuff defining an orifice; a telescoping airway channel, extending in a proximal direction from the cuff, defined by first and second concentric cylinders; and a spring that biases the first and second cylinders apart to extend the telescoping airway channel.

In an example, the first cylinder is an inner cylinder, and the second cylinder is an outer cylinder that slides along an outer surface of the first cylinder. In another example, the supraglottic airway device further includes an extension activator that is positioned on the outer cylinder. In a further example, the extension activator that is one of a button, pinch release, twist release, or slide release. In yet another example, the second cylinder is configured to receive an imaging extension of a video laryngoscope and be coupled to ventilation tubing.

In another aspect, the technology relates to a video airway assembly that includes a video laryngoscope having a camera stick with a camera at a distal end of the camera stick; and a supraglottic airway (SGA) device comprising an airway channel in communication with an orifice of a cuff and configured to transition from a contracted state to an extended state. When the camera stick is inserted into the airway channel while the SGA device is in the contracted state, the camera is positioned within the orifice of the cuff to image an airway of a patient; and when the camera stick is inserted into the airway channel while the SGA device is in the extended state, the camera does not reach the orifice of the cuff due to an extended length of the airway channel.

In an example, the SGA device further includes a first cylinder extending in a proximal direction from the cuff, the first cylinder defining a first lumen coupled to the orifice of the cuff; a second cylinder coupled to the first cylinder, wherein the second cylinder defines a second lumen that is substantially concentric with the first lumen, and the first lumen and the second lumen define the airway channel; and an extension activator that, when selected, causes the SGA device to extend from the contracted state to the extended state. In a further example, the first cylinder is an inner cylinder, and the second cylinder is an outer cylinder that slides along an outer surface of the first cylinder. In yet another example, the extension activator is one of a button, pinch release, twist release, or slide release. In still another example, the video airway assembly further includes a disposable sheath covering the camera stick, the sheath sized based on a diameter of the airway channel. In still yet another example, the sheath includes a lens positioned distally from the camera. In a further example, selection of the extension activator causes a spring to expand to extend the SGA device.

In another aspect, the technology relates to a method including receiving an imaging extension of an imaging device into an airway channel of a supraglottic airway (SGA) device; capturing, by the imaging device, an image of patient airways located distally from a cuff of the SGA device; receiving a selection of an extension activator of the SGA device while the SGA device is in a contracted state; in response to receiving the selection of the extension activator, extending a proximal portion of the SGA device to extend the airway channel and change the SGA device from the contracted state to an extended state; receiving ventilation tubing at a proximal end of the SGA device; and receiving breathing gases from the ventilation tubing into the airway channel.

In an example, extending the airway channel is caused by an outer cylinder sliding over an outer surface of an inner cylinder. In another example, the extension activator is one of a button, pinch release, twist release, or slide release.

It is to be understood that both the foregoing general description and the following Detailed Description are explanatory and are intended to provide further aspects and examples of the disclosure as claimed.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

Some of the figures included herein illustrate various embodiments of the disclosed technology from different viewing angles. Although the accompanying descriptive text may refer to such views as “top,” “bottom” or “side” views, such references are merely descriptive and do not imply or require that the disclosed technology be implemented or used in a particular spatial orientation unless explicitly stated otherwise.

FIG. 1 shows a schematic of a patient environment and airway management system including an example video airway assembly.

FIG. 2 shows a cross-sectional perspective view of the video airway assembly disposed in the patient of FIG. 1.

FIG. 3A shows a perspective view an example video airway assembly including a laryngeal mark airway and video laryngoscope.

FIG. 3B shows a perspective view of the laryngeal mask airway in FIG. 3A in an extended configuration.

FIG. 3C shows a perspective view of the laryngeal mask airway in FIG. 3A in a contracted configuration.

FIG. 3D shows a perspective view of the video laryngoscope in FIG. 3A.

FIG. 3E shows a perspective view of an alternative embodiment of the video laryngoscope and an example cover.

FIG. 4A is a flow diagram showing a process for operating a video airway assembly.

FIG. 4B provides a view of the example telescoping function of the laryngeal mask airway in FIG. 4A.

FIG. 5 is a flowchart showing an example method for operating a laryngeal mask airway for airway management procedures.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As disclosed briefly above, advanced airway management often involves use of specialized medical equipment (e.g., airway devices) to act as intermediaries or couplers between a patient and medical ventilator systems (i.e., patient interfaces). Supraglottic airway (SGA) devices, including laryngeal mask airways (LMAs), are a group of airway devices that are inserted into the pharynx to allow ventilation, oxygenation, and administration of anesthetic gases. Supraglottic airway devices are often relied upon in difficult airways to ventilate a patient after endotracheal intubation has repeatedly failed or as conduits to facilitate endotracheal intubation.

Generally, an LMA is an airway device comprising a hollow shaft or elongated tube connected to a mask-like cuff that is designed to sit in the hypopharynx facing the glottis, with the tip of the cuff at the esophageal inlet of a patient's oral cavity. The mask-like cuff of the laryngeal mask airway includes a ventilation orifice and a flexible or inflatable material to promote secure placement for ventilation via the ventilation orifice and to effectively seal the esophagus.

Blind intubation via some SGAs may fail either because of non-alignment of the device orifice and the glottis or because a tube or introducer (e.g., tracheal tube or catheter, respectively) passed down the laryngeal mask airway exits the ventilation orifice posteriorly and enters the esophagus. Some SGAs utilize visual guidance through the use of optics to increase the rate of successful intubation, in both normal and difficult airways of patients.

Visual guidance can help guide the laryngeal mask airway for proper placement but may cause the laryngeal mask airway to be dislodged upon removal and does not offer the advantages provided by video laryngoscopes such as an optical subassembly with a greater field of view, reusable or disposable elongated bodies, and covers or sheaths to enhance visualization and sterility (e.g., single-use) when inserted into an oral cavity of a patient.

Laryngoscopes are commonly used during intubation of a patient (e.g., an insertion of an endotracheal tube into a trachea of the patient). During intubation, the patient's airway and larynx may be visualized via direct laryngoscopy or indirect laryngoscopy. One form of indirect laryngoscopy is video laryngoscopy, which includes displaying a video image of the patient's larynx on a display screen. The video image displayed during video laryngoscope during an intubation procedure may assist a medical professional to visualize the patient's airway and facilitate manipulation and insertion of the endotracheal tube with the video laryngoscope. The display screen may be integrated into a video laryngoscope in a direct line-of-sight of the medical professional operating the video laryngoscope. Additionally or alternatively, a display screen may be a larger, external display separate from the housing of the video laryngoscope. An external display may allow other medical professionals participating in the medical procedure to track the progress of the intubation or other airway procedure in real time.

As discussed above, SGA insertion is generally performed blindly or using direct visualization, so placement can be challenging due to limited lighting, obstructed views, and poor attachability of the laryngeal mask airway cuff. In addition, improper SGA placement is generally difficult to detect. A poor seal may still allow some ventilation but the end of the SGA with gastric access may not be aligned. Problems may then potentially occur, such as when a patient aspirates while the gastric channel is pointed at vocal cords and the user does not know until unsuccessful attempt to suction. As such, SGAs would benefit from the enhanced visualization of video laryngoscopes to improve the efficiency and outcomes associated with laryngeal mask insertion.

Provided herein is an SGA incorporating the visualization advantages of video laryngoscopes to increase the likelihood of success or outcomes for patients with difficult airways. In one example, an SGA includes an elongated body, a plurality of tubular members concentrically disposed and forming the elongated body. The plurality of tubular members may include at least one inner cylinder and outer cylinder. The cylinders slide relative to one another to expand and contract the SGA. The expansion of the SGA may be performed automatically upon the selection of an expansion activator.

When the SGA is in the contracted state, a camera stick of a video laryngoscope may be inserted into the airway channel of the SGA to allow an operator to visually position and confirm the placement of the laryngeal mask airway. With the camera stick inserted into the airway channel, the additional rigidity provided by the camera stick may provide further control for the user to insert and position the SGA. When properly placed, the laryngeal mask airway is less likely to become dislodged from patient movement, which maintains the seal on the gastrointestinal tract of the patient and offers stable coupling or connecting with another device related to ventilation (e.g., ventilator) or subsequent airway procedures. When the visualization and positioning steps are complete, the SGA may be expanded and ventilation tubing may be coupled to the expanded SGA.

As used herein, the side toward which the device is inserted into a patient's airway is referred to as the “distal side” or “distal end,” and the side of an operator's proximal operation is referred to as the “proximal side” or “proximal end.” Likewise, “proximal” or “proximally” refers to the direction towards the operator and away from or up the patient's oral cavity and “distal” or “distally” refers to the direction of the patient's airway and towards or down the patient's oral cavity. A “contracted state” or “contracted configuration” is the state or configuration before extension of an extendable portion of the SGA. An “extended state” or “extended configuration” is the state or configuration before contraction of a contractable proportion of the SGA. While the description below is provided with the example of an LMA, the present technology may apply to other types of SGAs as well.

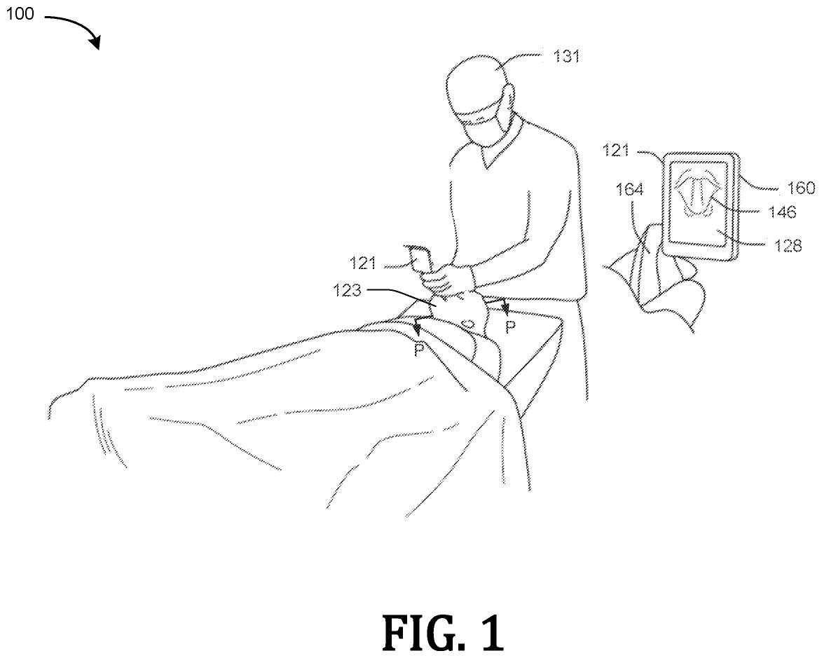

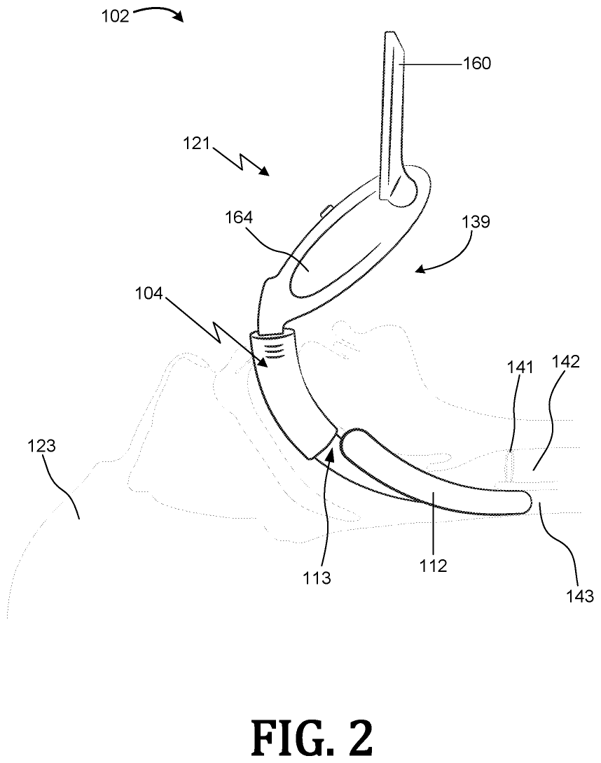

FIGS. 1 and 2 provide views of a patient environment 100 and video airway assembly 102 including a video laryngoscope 121 removably coupled with an LMA 104. The combination of the video laryngoscope and the LMA 104 may be referred to as a video airway assembly 102. The video airway assembly 102 may also refer generally to the assembly formed by coupling the LMA 104 with another type of optical airway device. FIG. 1 shows a schematic of a patient environment 100 and airway management system including an example video airway assembly 102 and a display 160 of the video airway assembly 102. FIG. 2 shows a cross-sectional perspective of the video airway assembly 102 disposed in the patient 123 of FIG. 1. Specifically, FIG. 2 depicts a side view of midsagittal plane (P-P) of a patient's head to show a side view of the LMA 104 when inserted into the patient 123.

In FIG. 1, the patient environment 100 may be any room where an airway management procedure is being performed, such as a medical suite in a hospital or other care setting, an operating or other procedure room, patient recovery room, an emergency setting, or other environments. As described herein, the video laryngoscope 121 may be inserted or coupled with the LMA 104 prior to positioning the LMA 104 in the patient 123 to provide greater airway visualization during such placement. Upon placing the LMA 104, the video laryngoscope 121 may also be used to verify that the LMA 104 is properly placed or positioned within an airway of the patient. For instance, the visualization may be used to confirm that a cuff 112 disposed at a distal portion 118 of the LMA 104 is covering or sealing the esophagus 143 and capable of providing breathable gas to the trachea 141 of the patient 123 (e.g., positioned in the patient's hypopharynx). In such a position, the LMA 104 isolates the trachea 142 for ventilation and reduces the risk of aspiration.

As shown in FIG. 1, a laryngoscope operator 131 (otherwise referred to herein as a clinician 131 or medical professional 131) holds a handle 164 of the video laryngoscope 121 coupled to a display portion 160 having a display screen assembly 128. The video laryngoscope 121 coupled with the LMA 104 may be used as part of an intubation procedure or other procedure to achieve ventilation. The video laryngoscope 121 may also be removed from the video airway assembly 102, and the airway channel of the LMA 104 may act as a conduit to carry breathing gasses or receive other insertable elements.

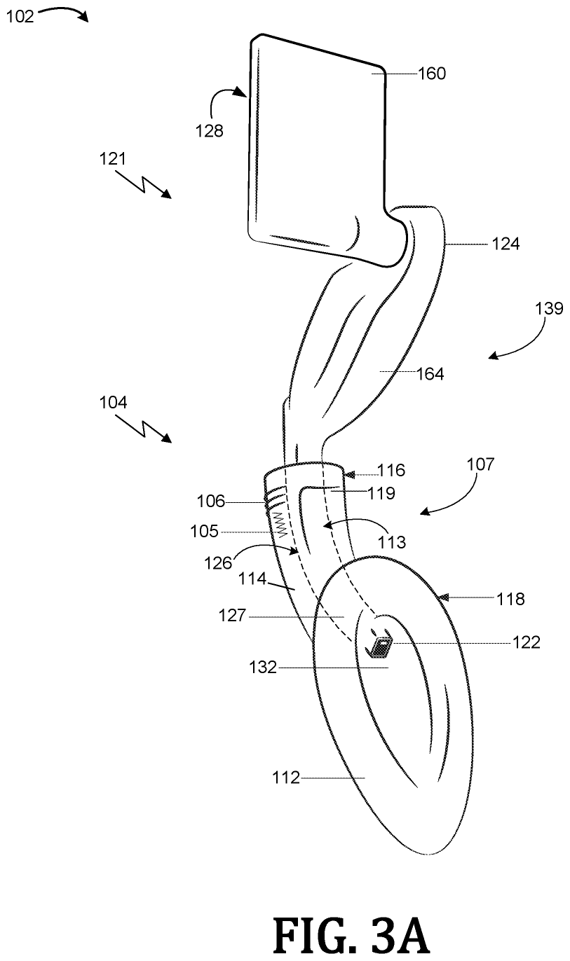

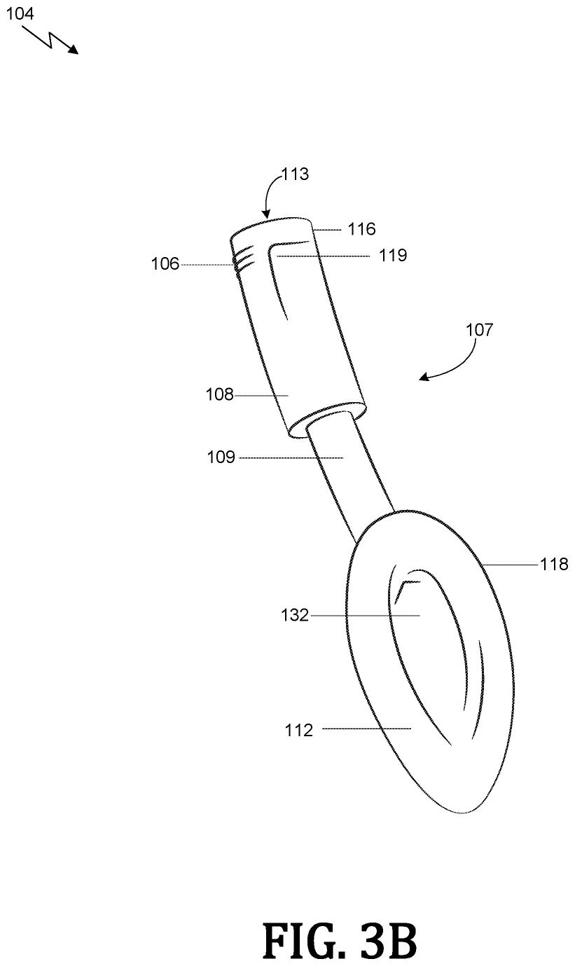

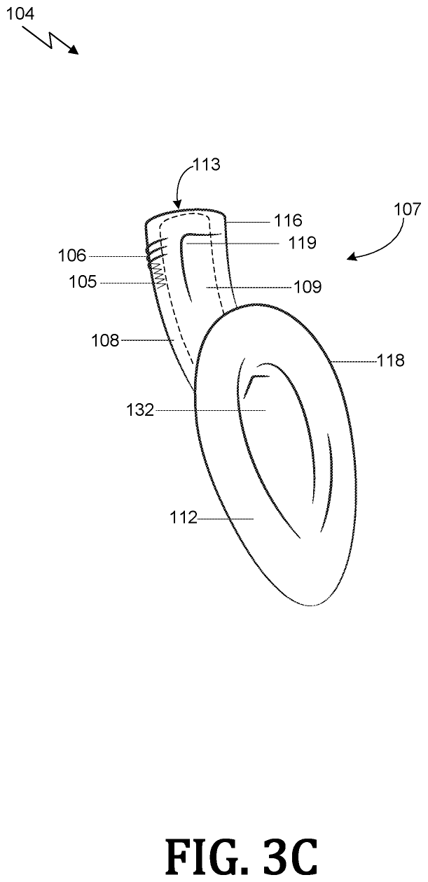

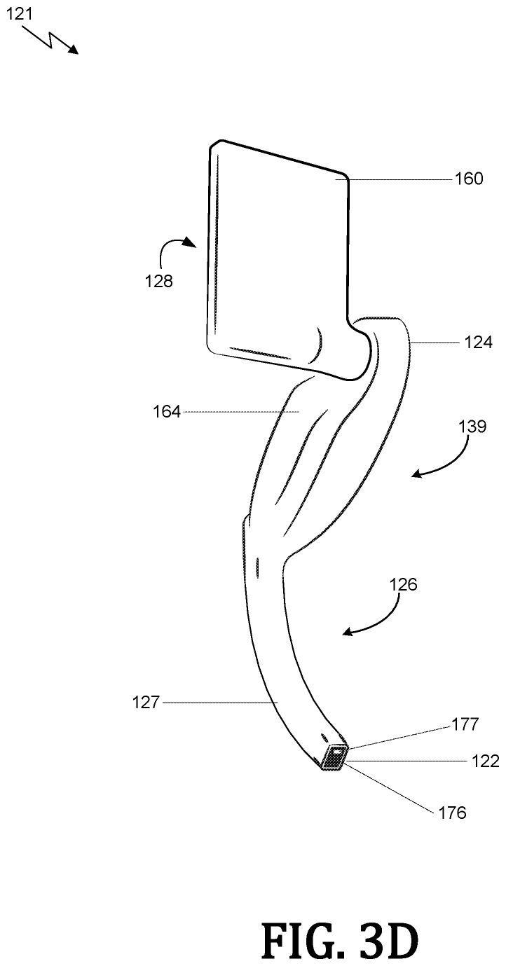

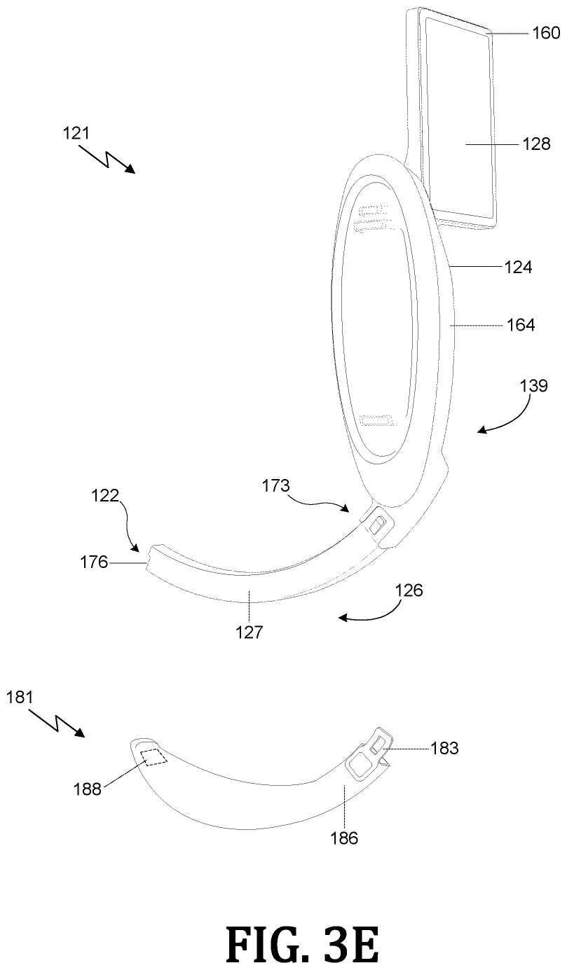

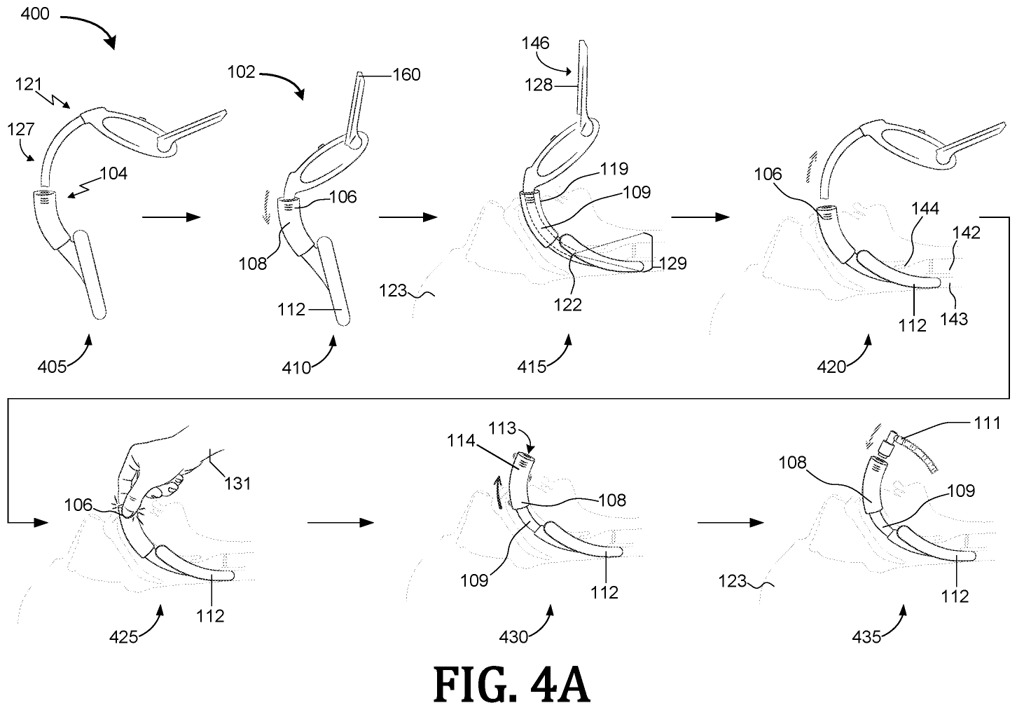

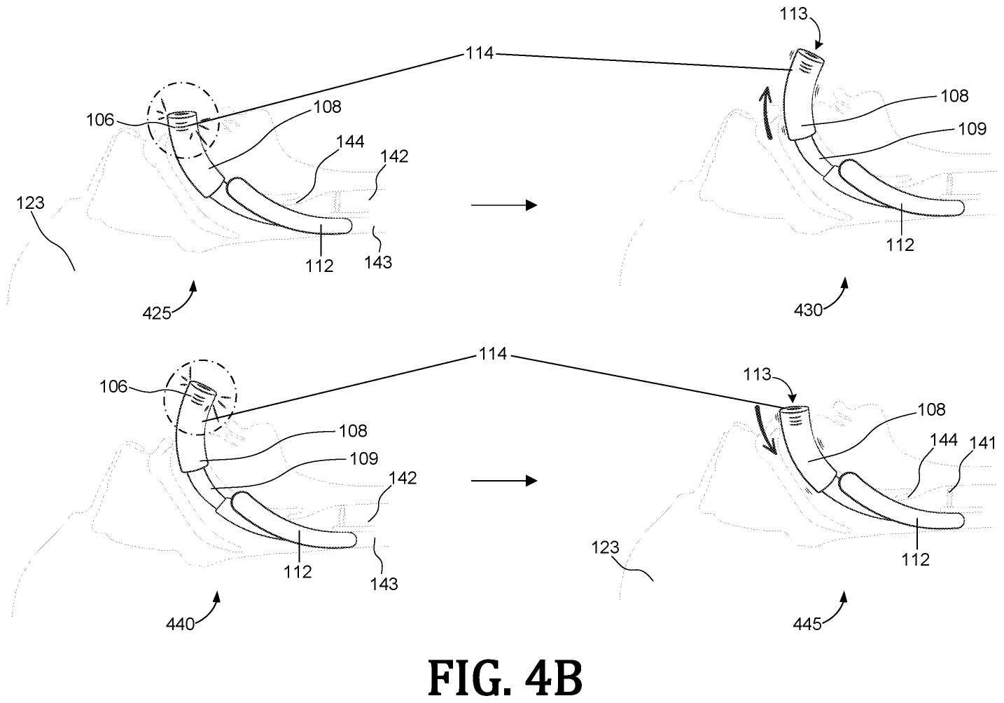

For example, the operator 131 may use the video laryngoscope 121 coupled with the LMA 104 in the patient's airway and uses the video laryngoscope 121 to confirm proper placement of the LMA 104. Upon confirmation, the operator removes the video laryngoscope 121 from the LMA 104 secured in the patient's airway (e.g., over the larynx). After removing the video laryngoscope 121 from the placed LMA 104 of the video airway assembly 102, the operator may establish or re-establish an airway to ventilate the patient by connecting the airway channel 113 of the placed LMA 104 with a ventilation system (e.g., ventilator tubing or connectors). Further aspects of the video airway assembly 102, and additional capabilities are provided in FIGS. 3A, 4A, and 4B and their associated descriptions below.

Video images 146 may be acquired by the video laryngoscope 121 during placement of the LMA 104. The acquired images 146 are displayed on one or more display screens. For example, as shown in FIG. 1, the acquired images 146 may be displayed at an integrated display (e.g., display assembly 128) of the video laryngoscope 121 in the line-of-sight of the operator 131. The images 146 acquired and displayed to the operator 131 may correspond to a field of view 129 of the optical subassembly 122 at various stages of an airway procedure. The displayed images may also be displayed on other monitors to allow for other clinicians in the patient environment 100 to assist the operator 131, monitor a condition of the patient, prepare or adjust medical equipment in the patient environment, and/or wait until the LMA 104 is placed and maintaining the patient's airway to perform other procedures or interventions.

FIG. 2 provides a view of the video laryngoscope 121 and the LMA 104 when inserted into the airway of the patient 123. The cross-sectional perspective of the patient 123 is taken along the line P-P in FIG. 1, which shows the video airway assembly 102 and its associated components disposed in the patient 123. As discussed further below, the video airway assembly 102 includes the LMA 104, which is configured to move between a contracted configuration and an extended configuration. The LMA 104 includes an airway channel 113, and an insertable imaging extension (e.g., camera stick) of the video laryngoscope 121 slides into the airway channel 113 of the LMA 104. The LMA 104 and video laryngoscope 121 of the video airway assembly 102 are removably coupled to each other upon inserting the insertable section of the video laryngoscope 121 into the LMA 104. In some examples, the example video airway assembly 102 is formed prior to intubation of the patient 123, and after formation of the video airway assembly 102, the operator may proceed with advancing the LMA 104 of the video airway assembly 102 into the patient's airway to perform the intubation.

FIG. 3A shows a perspective view an example video airway assembly 102 including a LMA 104 and video laryngoscope 121. The LMA 104 includes a body 107 (e.g., reusable body) that includes an upper or proximal portion 116, an airway channel 113, and a distal or lower portion 118. The proximal portion 116 may include a bite block 119 that is configured to protect the patient's teeth and mouth when inserting and placing the LMA 104.

The proximal portion 116 also includes an extension activator 106 that, when pressed or selected, causes or allows a telescoping segment 114 to extend. For instance, the extension activator 106 may be a button, pinch/squeeze release, twist release, slide release, or other similar mechanisms that allow the telescoping segment 114 to move (e.g., be released). In some examples, the LMA 104 may include a spring 105 or similar structure that stores energy (e.g., in the form of potential energy) when the LMA 104 is in the contracted state. Upon the extension activator 106 being released, the spring 105 or similar structure releases the energy (e.g., in the form of kinetic energy) to cause the telescoping segment 114 to extend and the LMA 104 to be in the extended state. Accordingly, the LMA 104 may extend in a manner that does not require the operator to pull on the proximal end of the LMA 104, which could dislodge the LMA 104 from its proper positioning.

The distal portion 118 of the LMA 104 includes a cuff 112 that may be configured to seal the esophagus 143 of the patient 123 when placed by a medical practitioner (e.g., operator 131) during an airway management procedure. The cuff 112 may be incorporated on the distal portion 118 of the LMA 104 and selectively inflatable to securely place the LMA 104 at the desired position within the patient's airway.

The LMA 104 may include multiple tubes (e.g., cylinders) extending from the proximal portion 116 to the distal portion 118 of the body 107 to define at least one shared channel or lumen therein, which may be referred to as the airway channel 113. The airway channel 113 is configured to receive an insertable section, such as camera stick 127, of the video laryngoscope 121 such that the distal section 126 of the video laryngoscope 121 is contiguous with the distal portion 118 of the LMA 104 and capable of viewing the larynx of the patient 123. For instance, the distal end of the camera stick 127 includes an optical subassembly 122 (e.g., camera) that can be contiguous or slightly protrude from an opening (e.g., ventilation orifice 132) of the cuff 112 disposed at the distal portion 118 of the LMA 104.

The optical subassembly 122 may be operatively connected to a display screen assembly 128 of the video laryngoscope 121. The display screen assembly 128 is integrated with or connected to a body 139 of the video laryngoscope 121 at a proximal section 124. The display assembly 128 may include a display screen which may be rotated about the handle 164 (such as around a pivot point or hinge) to change the viewing angle of the screen by the clinician.

In some embodiments, the optical subassembly 122 and/or distal portion 118 of the LMA 104 may include a lens. The lens may alternatively or additionally be part of a disposable sheath or cover (e.g., sheath 181 in FIG. 3E) that is configured to mate with and surround the insertable section of the video laryngoscope 121. In this way, the insertable section and sheath mated therewith are simultaneously received by the airway channel 113 of the LMA 104. The optical subassembly 122, adjacent the lens, is operable to capture images that are displayed on the screen 128 and thereby provide an indirect view of a patient's oral cavity (and in particular the epiglottis, trachea or larynx) in use. An inferior surface of LMA 104 which, in use, may engage with a patient's tongue. The inferior surface of the embodiments shown is provided with a moderate curvature, so as to enable the operator 131 of the integrated laryngeal mask airway and video laryngoscope (e.g., video airway assembly 102) to directly view the patient's airway in proximity to the cuff 112 at the laryngeal mask airway's distal portion 118, and thus obtain a direct view of a patient's airways.

The proximal end 116 of the airway channel 113 may be provided with a removable friction-fit connector (not shown) designed for attachment to conventional anesthetic or breathing tubes so that the LMA 104 may be used in a stand-alone manner to treat or ventilate the lungs of a patient. For instance, when the video laryngoscope 121 is removed from the LMA 104, ventilation tubing attached to a ventilator may be connected to the proximal end 116 of the airway channel to provide breathing gases to the patient through the LMA 104.

The bite block 119 may be integral with the laryngeal mask airway and serves various functions, including contributing to the stability of the laryngeal mask airway during insertion and it also helps in providing good vertical stability and axial strength during insertion. The bite block 119 is capable of withstanding a force greater than the human bite. The bite block may be integrated with outer cylinder of the laryngeal mask airway and available in multiple sizes that correspond to the circumference of the outer cylinder of the laryngeal mask airway. Additionally, the airway channel 113 may include the bite block 119 designed to inhibit the patient from biting the inserted assembly occupying the airway channel 113 (e.g., video laryngoscope 121) or otherwise collapsing the airway channel 113.

In the example depicted in FIG. 3A, the LMA 104 is shown in its contracted configuration. FIG. 3B depicts the LMA 104 in its extended configuration without the video laryngoscope 121, and FIG. 3C depicts the LMA 104 in its contracted configuration without the video laryngoscope 121. The mechanisms for providing the telescoping functionalities of the LMA 104 can be more easily seen in FIGS. 3B-3C.

In some examples, the telescoping functionality is achieved through concentric tubes or cylinders that slide relative to one another. For instance, the concentric cylinders may include an outer cylinder 108 positioned at the proximal end 116 and an inner cylinder 109 positioned distally from the outer cylinder 108 when the LMA 104 is in the extended configuration. The inner cylinder 108 defines a first lumen that is coupled or in pneumatic communication with the orifice of the cuff 112. The outer cylinder 108 defines a second lumen that is substantially concentric with the first lumen, and the first lumen and the second lumen define the airway channel 113.

The outer cylinder 108 is movable with respect to the inner cylinder 109 such that an interior surface of the outer cylinder 108 slides against the exterior or outer surface of the inner cylinder 109 when the LMA 104 is extended into its extended configuration. In the extended configuration, the airway channel 113 is formed of an inner lumen defined by the outer cylinder 108 and an inner lumen defined by the inner cylinder 109. In other examples, the outer and inner cylinders may be switched. For instance, in the extended configuration, an inner cylinder may extend proximally from an outer cylinder that is attached to the cuff 112. In either case, when the LMA 104 transitions from the contracted state to the expanded state, the airway channel 113 of the LMA 104 extends. In general, the segment of the LMA 104 that moves away from the cuff may be referred to herein as the telescoping segment 114. In the example depicted, the telescoping segment 114 is the outer cylinder 108.

Extending the outer cylinder 108 from the inner cylinder 109 may be accomplished without movement of the distal end of the LMA 104, such as the cuff 112. Accordingly, the LMA 104 may be extended after the LMA 104 has been positioned without risking that the LMA 104 may become dislodged from its position within the patient's airway.

The extension of the LMA 104 from the contracted configuration to the extended configuration may be triggered through the selection or interaction with the extension activator 106. Selection or interaction with the extension activator 106 causes or allows the outer cylinder 108 to be extended proximally from the inner cylinder 109. For example, when the LMA 104 is in the contracted configuration, the telescoping actuator in a primed or biased state, such that activating a telescoping actuator (e.g., spring 105) causes the proximal portion (e.g., outer cylinder 108) to move relative to the body 107 of the LMA 104. The structure and extension and/or contraction features of the telescoping segment 114 are shown and described in greater detail with reference to FIGS. 4A and 4B.

FIG. 3D shows the example video laryngoscope of FIG. 3A. FIG. 3D provides a perspective view of the video laryngoscope 121 uncoupled from the video airway assembly 102 shown in FIG. 3A.

As shown, the video laryngoscope 121 has a body 139 (e.g., reusable body). The body 139 includes a display portion 160 having a display screen assembly 128 that is configured to display images and/or other data, a handle portion 164 having a handle that is configured to be gripped by the medical professional during the airway management procedure, and an elongate portion or arm 127 that supports an optical subassembly 122, such as camera 176 and light source 177 (e.g., light-emitting diodes (LEDs)), that is configured to obtain images (e.g., acquired images 146, which may be still images and/or moving images, such as videos). The camera 176 and light source 177 of the optical subassembly 122 may be incorporated on the distal end of the arm 127. In some examples, the arm 127 may be referred to as a camera stick 127. The light source 177 may be provided as part of the camera 176 or separate from the camera 176 on the arm 127. The captured images may be displayed on a display screen assembly 128 of the display portion 160.

FIG. 3E depicts another example of the video laryngoscope 121. As shown, the display portion 160 and the handle portion 164 may not be distinct portions, such that the display screen assembly 128 is integrated into or coupled the handle portion 164. In the illustrated example, a removable laryngoscope sheath 181 (e.g., disposable cover, sleeve, or blade) is positioned over the arm 127 to help protect the arm from contamination during use.

In some examples, the sheath 181 may be attached and detached from the video laryngoscope 121 through mating connectors. For instance, a male connector 173 disposed adjacent the arm 127 of the video laryngoscope 121 is configured to mate with a female connector 183 disposed at a proximal end 186 of the sheath 181. The sheath 181 may also be attached through other types of elements, or through a slide or friction fit. Accordingly, the sheath 181 is configured to be removably attached to an insertable section (e.g., arm of video laryngoscope) before coupling the insertable section and attached blade with the laryngeal mask airway for an airway management procedure. The sheath 181 may be single-use and disposable to limit contamination of the video airway assembly from contact with the patient's mouth or other unsterile surface.

The sheath 181 may also serve to enhance visualization by including a lens 188 for an optical subassembly of the insertable section. For instance, when the sheath 181 is fully attached to the arm 127, the lens aligns with the camera 176 to provide improved or adjusted imaging of the airways.

The size and shape of the sheath 181 may be selected based on the configuration of the LMA 104, such as the size of the airway channel 113. For instance, the sheath 181 may be used to expand or increase the circumference of the insertable section (e.g., camera stick 127) such that when inserted into an airway channel 113 of the LMA 104, the insertable section forms a sliding fit with the laryngeal mask airway by snuggly occupying the lumen of the airway channel 113. In some examples, multiple sizes of the sheaths 181 may be provided for use with different types of LMAs. The fit between the sheath 181 and the airway channel 113 of the LMA 104 may provide some resistance to movement, but the fit may still allow for the sheath 181 (and the camera stick 127) to be easily removed from the airway channel 113 without dislodging the LMA 104. In other examples, the camera stick 127 may be inserted into the LMA 104 without the use of the sheath 181.

FIG. 4A is a flow diagram 400 showing a process for operating a video airway assembly 102. As discussed above, the video airway assembly 102 refers to the assembly formed upon coupling of the LMA 104 and the video laryngoscope 121. The video airway assembly 102 may also refer generally to the assembly formed by coupling the LMA 104 with another optical airway device.

FIG. 4A depicts multiple stages 405-435 of the video airway assembly 102 being inserted into a patient during a procedure. At stage 405, the video laryngoscope 121 is inserted into the airway channel 113 of the LMA 104. More specifically, the camera stick 127 is inserted into the airway channel 113 of the LMA 104 when the LMA 104 is the contracted state. In some examples, a sheath 181 is first attached to the camera stick 127 prior to insertion into the LMA 104. Stage 410 shows the video laryngoscope 121 inserted into the LMA 104 to form the video airway assembly 102 that is ready to be placed in a patient (e.g., patient 123).

At stage 415, the formed video airway assembly 102 is placed within the airway of the patient 123 (e.g., at the epiglottis 144). The optical subassembly 122 at the distal section 126 of the video laryngoscope 121 may then be used to confirm the placement of the video airway assembly 102. As shown, the field of view 129 provides an unobstructed view of the patient's larynx such that navigation and proper placement of the video airway assembly 102 is assured. For instance, when the LMA 104 is in the contracted state, the camera of the video laryngoscope is positioned within the orifice of the cuff to image the airways of the patient.

At stage 420, upon properly positioning the LMA 104, the video laryngoscope 121 may be removed or decoupled from the LMA 104. Removal of the video laryngoscope 121 does not cause the body 107 of the LMA 104 to be displaced or dislodged from its placement in the airway of the patient 123. In some examples, the operator may hold the LMA 104 during the removal process to help prevent movement of the LMA 104.

At stage 425, the telescoping segment 114 of the LMA 104 can be engaged by the operator interacting with the extension activator 106 at the proximal portion of the LMA 104. In the example depicted, in response to selection of the extension activator 106, an outer cylinder or tube 108 of the body 107 of the LMA 104 extends away from the patient 123 (e.g., extends in a proximal direction). Various structural features can be incorporated into the LMA 104 that automatically extend the outer cylinder 108 of the LMA 104. For example, a spring element primed or biased in the contracted configuration of the LMA 104 may be activated or released upon the operator selecting the extension activator 106. At stage 430, the telescoping segment 114 extends proximally away from the cuff 112, where the LMA 104 is then in the extended configuration. If the camera stick of the video laryngoscope 121 was to be inserted into the LMA 104 when in the extended configuration, the camera would not reach the orifice of the cuff due to the extended length of the airway channel 113.

At stage 435, the proximal portion of the extended configuration of the LMA 104 may be connected to ventilation tubing 111. Breathing gases may then be delivered to the patient 123 via the cuff 112 of the properly positioned LMA 104. Following stage 435, the stages 405-435 may be reversed if the clinician desires to view the airways via the video laryngoscope 121 at a later point in time.

FIG. 4B provides a diagram of the of the LMA 104 changing between contracted states and extended states. Stages 425 and 430 of FIG. 4A are shown again in FIG. 4B at a larger scale. At stage 425, the initial state of LMA 104 is the contracted configuration. In this configuration, activation of extension activator 106 causes the telescoping segment 114 (e.g., the outer cylinder 108) of the LMA 104 to proximally extend away from the cuff 112 as well as the patient 123. In the depicted example, the outer cylinder 108 slides along the inner cylinder 109.

In some examples, the telescoping segment 114 may be configured to both extend as well as contract. For instance, a second selection of the extension activator may cause the outer cylinder 108 of the LMA 104 to also slide distally back to towards the cuff 112, and the LMA 104 returns to its contracted state. The contraction may be performed through manual force by the operator automatically through an actuator in the LMA 104 depending on the example.

For instance, at stage 440, the LMA is in the extended state. When in the extended state, the telescoping segment may be locked or fixed in the extended state. For example, a latch, detent, or similar mechanism may included in the outer cylinder 108 or the inner cylinder 109 such that when the outer cylinder 108 is in the extended state, the outer cylinder is fixed in that position until a subsequent selection of the extension activator 106. The selection of the extension activator releases the outer cylinder 108 from its fixed position, such as by releasing the latch, withdrawing the detent, etc. The outer cylinder 108 may then be slid back towards the cuff, as shown in stage 445.

At stage 445, the LMA 104 is in the contracted state. When in the contracted state, the outer cylinder 108 may also be locked or fixed in the state by a latch, detent, or similar mechanism. The sliding of the outer cylinder 108 back towards the cuff 112 may also compress a spring or similar element to cause energy to be stored in the spring. Then, when the extension activator 106 is subsequently selected, the outer cylinder 108 is released and the spring releases the stored energy to extend the outer cylinder away from the cuff.

Various types of actuators may be used to enable the telescoping functions of the LMA 104. For example, mechanical, electrical, hydraulic, pneumatic and/or combinations of these actuator types may be integrated with the body 107 of the LMA 104. Furthermore, the actuator integrated with the LMA 104 can be single-action or double-action in accordance with various embodiments. The outer cylinder 108 of the body 107 of the LMA 104 is moved without changing its rotational orientation through the use of linear actuators.

Alternatively, the telescoping mechanism, for example, can describe a click and lock mechanism akin to the operating principle in a ballpoint pen. In such an example, the telescoping mechanism may include a cam body, tubular plunger, stop members, and spring elements. The cam body is affixed to the proximal portion 116 and the spring elements are diametrically opposed within the body 107 of the LMA 104. The LMA 104 can be in either of two positions or configurations: the contracted configuration and the extended configuration. In the contracted configuration, the outer cylinder 108 circumscribes the inner cylinder 109 such that the inner cylinder 109 is telescopically disposed within the outer cylinder 108 (i.e., inner cylinder is not visible). In the extended configuration, the outer cylinder 108 proximally extends relative to the position of the inner cylinder 109 such that only an end of the inner cylinder 108 is telescopically disposed within the outer cylinder 108 (i.e., majority of inner cylinder 109 is visible).

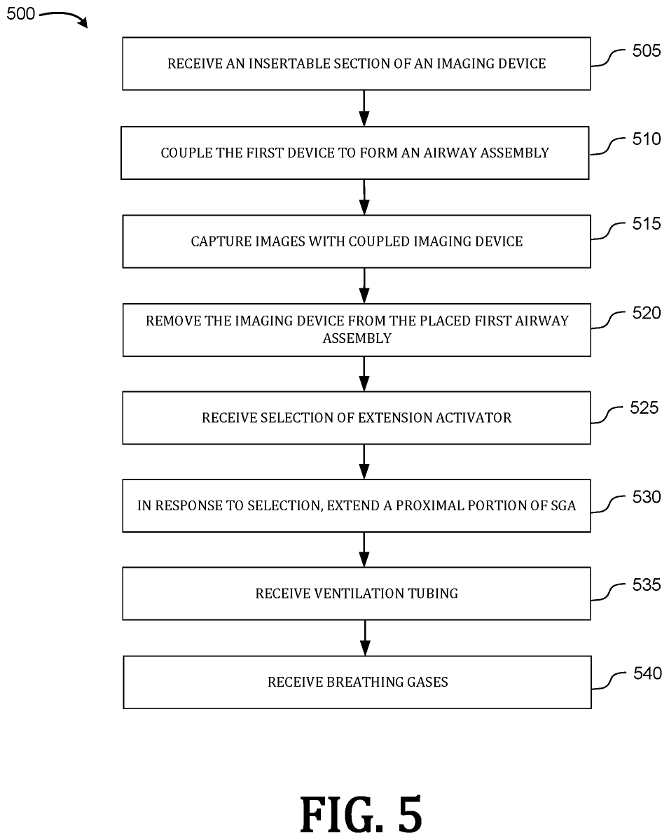

FIG. 5 shows an example method 500 according to the disclosed technology. The example method includes operations that may be implemented or performed by the systems and devices disclosed herein. For example, the video laryngoscope 121/221, the LMA 104, and/or video airway assembly 102 depicted in at least FIGS. 2, 3A, 4A, and 4B may perform the operations described in the methods.

The method 500 provides an example process for using a laryngeal mask airway that can integrate with various other imaging devices (or components thereof) to facilitate airway support. At operation 505, a laryngeal mask airway (e.g., LMA 104) receives an insertable imaging extension (e.g., camera stick) or assembly of an imaging device, such as a video laryngoscope. The insertable imaging extension of the first device may be inserted at the proximal or upper portion 116 of the LMA 104 such that it occupies the airway channel 113 within the body 107 of the LMA 104. For instance, a camera stick of a video laryngoscope is received into the airway channel. In some examples, the circumference of an insertable imaging extension of the imaging device may be modified such that the insertable imaging extension fits more tightly into the airway channel 113 (e.g., addition of sheath 181).

At operation 510, the insertable imaging extension (e.g., distal end) of the imaging device received by the LMA 104 may be removably coupled or attached to the body 107 of the LMA 104. The insertable imaging extension and airway channel 113 may couple with each other via corresponding mating connectors (i.e., male and female connectors), latches, and/or protrusions disposed in the body 107 of the LMA 104 and/or the insertable imaging extension. In other examples, sufficient coupling may be achieved without additional coupling mechanisms (e.g., friction fit). As such, the fit between the insertable imaging extension of the first device and the laryngeal mask airway forms an integrated airway assembly that can move as a unit when inserted into the oral cavity of a patient. In the example of the first device being the video laryngoscope 121, the combination of the video laryngoscope 121 and LMA 104 forms the video airway assembly 102.

At operation 515, images are captured by the inserted portion of the imaging device of the airway assembly. For instance, the images may be captured by an optical subassembly 122 of the video laryngoscope 121. The captured images may be displayed on a screen of the video laryngoscope 121 to guide the placement of the LMA 104.

At operation 520, upon secure attachment and positioning of the laryngeal mask airway, the imaging device be removed or released from the video airway assembly 102 without dislodging the properly placed laryngeal mask airway. For example, the imaging device may be removed or released from the upper portion of the laryngeal mask airway by releasing the latch or protrusion used to couple the imaging device to the LMA.

At operation 525, a selection of the extension activator is received. The selection may be in the form of a push, pull, twist, pinch, squeeze, etc. depending on the particular type of extension activator and corresponding actuator that is utilized in the LMA 104. At operation 530, the telescoping segment (e.g., the outer cylinder 108) extends proximally in response to the selection of the extension activator. The extension may occur automatically by the actuator releasing stored energy, such as the expansion of a spring.

At operation 535, ventilation tubing is coupled to the proximal end of the LMA 104 in its extended state. At operation 540, breathing gases from a ventilator is received via the ventilation tubing.

Although the present disclosure discusses the implementation of these techniques in the context of an airway assembly capable of effectively accessing and securing a patient's airway for ventilation and/or intubation, the techniques introduced above may be implemented for a variety of medical devices or devices related to restoring a patient's airway. A person of skill in the art will understand that the technology described in the context of an optical laryngeal mask airway for human patients could be adapted for use with other systems such as laryngeal mask airways for non-human patients, general airway management systems, and/or medical video imaging systems. Additionally, a person of ordinary skill in the art will understand that the telescoping mechanism may be implemented by a variety of actuators and associated structural components.

Although this disclosure describes a video airway assembly comprising a laryngeal mask airway and video laryngoscope, other implementations include airway devices lacking optics that are capable of receiving insertable imaging extensions or assemblies via a main lumen or channel and require navigating a patient's airway. In these implementations, the telescoping mechanism and related structural components (i.e., movable concentric cylinders) may be incorporated to provide similar video and modular capabilities as those described herein.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A telescoping supraglottic airway device, comprising:
a cuff defining an orifice;
a telescoping airway channel, extending in a proximal direction from the cuff, defined by first and second concentric cylinders; and
a spring that biases the first and second cylinders apart to extend the telescoping airway channel.

2. The supraglottic airway device of claim 1, wherein the first cylinder is an inner cylinder, and the second cylinder is an outer cylinder that slides along an outer surface of the first cylinder.

3. The supraglottic airway device of claim 2, further comprising an extension activator that is positioned on the outer cylinder.

4. The supraglottic airway device of claim 3, wherein the extension activator that is one of a button, pinch release, twist release, or slide release.

5. The supraglottic airway device of claim 1, wherein the second cylinder is configured to receive an imaging extension of a video laryngoscope and be coupled to ventilation tubing.

6. A video airway assembly, comprising:
a video laryngoscope having a camera stick with a camera at a distal end of the camera stick; and
a supraglottic airway (SGA) device comprising an airway channel in communication with an orifice of a cuff and configured to transition from a contracted state to an extended state, wherein:
when the camera stick is inserted into the airway channel while the SGA device is in the contracted state, the camera is positioned within the orifice of the cuff to image an airway of a patient; and
when the camera stick is inserted into the airway channel while the SGA device is in the extended state, the camera does not reach the orifice of the cuff due to an extended length of the airway channel.

7. The video airway assembly of claim 6, wherein the SGA device further comprises:
a first cylinder extending in a proximal direction from the cuff, the first cylinder defining a first lumen coupled to the orifice of the cuff;
a second cylinder coupled to the first cylinder, wherein the second cylinder defines a second lumen that is substantially concentric with the first lumen, and the first lumen and the second lumen define the airway channel; and
an extension activator that, when selected, causes the SGA device to extend from the contracted state to the extended state.

8. The video airway assembly of claim 7, wherein the first cylinder is an inner cylinder, and the second cylinder is an outer cylinder that slides along an outer surface of the first cylinder.

9. The video airway assembly of claim 7, wherein the extension activator is one of a button, pinch release, twist release, or slide release.

10. The video airway assembly of claim 7, further comprising a disposable sheath covering the camera stick, the sheath sized based on a diameter of the airway channel.

11. The video airway assembly of claim 10, wherein the sheath includes a lens positioned distally from the camera.

12. The video airway assembly of claim 7, wherein selection of the extension activator causes a spring to expand to extend the SGA device.

13. A method comprising:
receiving an imaging extension of an imaging device into an airway channel of a supraglottic airway (SGA) device;
capturing, by the imaging device, an image of patient airways located distally from a cuff of the SGA device;
receiving a selection of an extension activator of the SGA device while the SGA device is in a contracted state;
in response to receiving the selection of the extension activator, extending a proximal portion of the SGA device to extend the airway channel and change the SGA device from the contracted state to an extended state;
receiving ventilation tubing at a proximal end of the SGA device; and
receiving breathing gases from the ventilation tubing into the airway channel.

14. The method of claim 13, wherein extending the airway channel is caused by an outer cylinder sliding over an outer surface of an inner cylinder.

15. The method of claim 13, wherein the extension activator is one of a button, pinch release, twist release, or slide release.
